(19) United States
(12) Patent Application Publication
(10) Pub. No.: US 2011/0098601 A1
(43) Pub. Date: Apr. 28, 2011

(54) MEDICAL DEVICE ASSEMBLY HAVING FREEDOM OF ROTATION

(76) Inventors: Ky Huynh, Tigard, OR (US); Kirk Mikkelsen, Chaska, MN (US)

(21) Appl. No.: 12/912,661

(22) Filed: Oct. 26, 2010

Related U.S. Application Data
(63) Continuation-in-part of application No. 12/606,150, filed on Oct. 26, 2009.

(51) Int. Cl. A61B 18/00; H01R 39/00; A61N 7/00; A61B 5/05

(52) U.S. Cl. 600/585; 439/18; 606/41; 604/22; 606/45

(57) ABSTRACT

A method of performing a surgical procedure using an electrical surgical device, which utilizes an electrical surgical assembly including the surgical device connected to a base station by a cable having a plurality of mutually electrically insulated conductors, and an electrical adapter interposed at a point between the base station and the device. The adapter is comprised of a first half and a second half that have freedom of rotation relative to each other. The first half is equipped with a first connector-half and the second half is equipped with a second connector-half. While the surgical procedure is performed, the adapter permits relative rotation between the first half and the second half, thereby avoiding a problem of cable twisting.

### RELATED APPLICATIONS

This application is a continuation-in-part of application Ser. No. 12/606,150 filed Oct. 26, 2009.

### BACKGROUND

Increasingly, hand held medical devices are connected to a base station with a multi-connector cable ferrying data from the device and commands to the device. Diagnostic or therapeutic catheters, for example, ablation catheters, ultrasound imaging (IVUS) catheters and electrophysiology mapping catheters, all produce data that must be delivered to a base station and may require commands from a base station. Unfortunately, medical devices must often be manipulated by a medical professional who is concentrating deeply about the task at hand. The manipulation may cause a rotation of the device. As a result the cable for the medical device becomes twisted, resisting further rotation and thereby interfering with the procedure. Potential bending of the catheter threatens data and power flow and could harm the cable.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

In a first separate aspect, the present invention may take the form of a method of performing a surgical procedure using an electrical surgical device, which utilizes an electrical surgical assembly including the surgical device connected to a base station by a cable having a plurality of mutually electrically insulated conductors, and an electrical adapter interposed at a point between the base station and the device. The adapter is comprised of a first half and a second half that have freedom of rotation relative to each other. The first half is equipped with a first connector-half and the second half is equipped with a second connector-half. While the surgical procedure is performed, the adapter permits relative rotation between the first half and the second half, thereby avoiding a problem of cable twisting.

In a second separate aspect, the present invention may take the form of an electrical medical device assembly that includes a handheld unit, adapted to be manipulated by a medical professional and requiring multi-conductor electrical connection to a base unit and a base unit adapted to provide electrical power to the handheld unit. Also, a multi-conductor electrical cable connects the handheld unit to the base unit and a multi-conductor electrical adapter is interposed between the handheld unit and the base unit, the adapter including a first half and a second half and wherein relative rotation is permitted between the first half and the second half and wherein the first half is equipped with a first connector-half and the second half is equipped with a second connector-half.

In a third separate aspect, the present invention may take the form of an adapter that includes a first half that defines a plurality of contacts having circular conductive surfaces and a second half that includes a set of resilient contacts, each positioned to contact one of the circular conductive surfaces to create an electrical connection.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced drawings. It is intended that the embodiments and figures disclosed herein are to be considered illustrative rather than restrictive.

FIG. 1 is a diagram of a medical device assembly that may be implemented in accordance with the present invention.

FIG. 2A is a top side perspective view of a connector according to the present invention, in closed form.

FIG. 2B is a top side perspective view of the connector of FIG. 1A, in open form.

FIG. 2C is a side sectional view of the connector of FIG. 1A taken along line 1C-1C of FIG. 1A.

FIG. 2D is an alternative preferred embodiment of the connector of the present invention, which is internally the same as the connector of FIG. 1A, but which is embedded into a base station.

FIG. 3A is a top side perspective view of an alternative preferred embodiment of a connector according to the present invention, shown in closed form.

FIG. 3B is a top side perspective view of the connector of FIG. 2A, shown in open form.

FIG. 4A is a top side perspective view of a connector according to an alternative preferred embodiment of the present invention, shown in closed form.

FIG. 4B is a top side perspective view of the connector of FIG. 4A, shown in open form.

FIG. 5 is a side perspective view of an multi-conductor adapter, relative rotation permitting adapter, according to a preferred embodiment of the present invention.

FIG. 6 is a perspective view of a part of the adapter of FIG. 5, expanded relative to the view of FIG. 5.

FIG. 7 is a cross-sectional view of the part of FIG. 6, taken along line 7-7 of FIG. 6.

FIG. 8 is a cut-away view of the part of FIG. 6.

FIG. 9 is an exploded cut-away view of the part of FIG. 6.

### DETAILED DESCRIPTION OF THE PREFERRED EMBODIMENTS

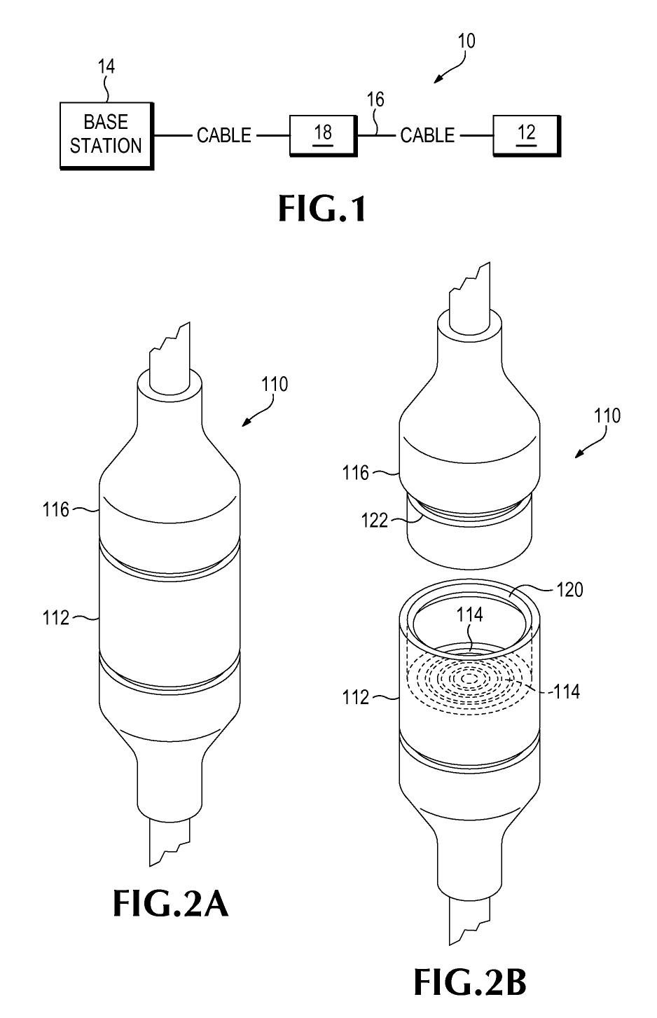

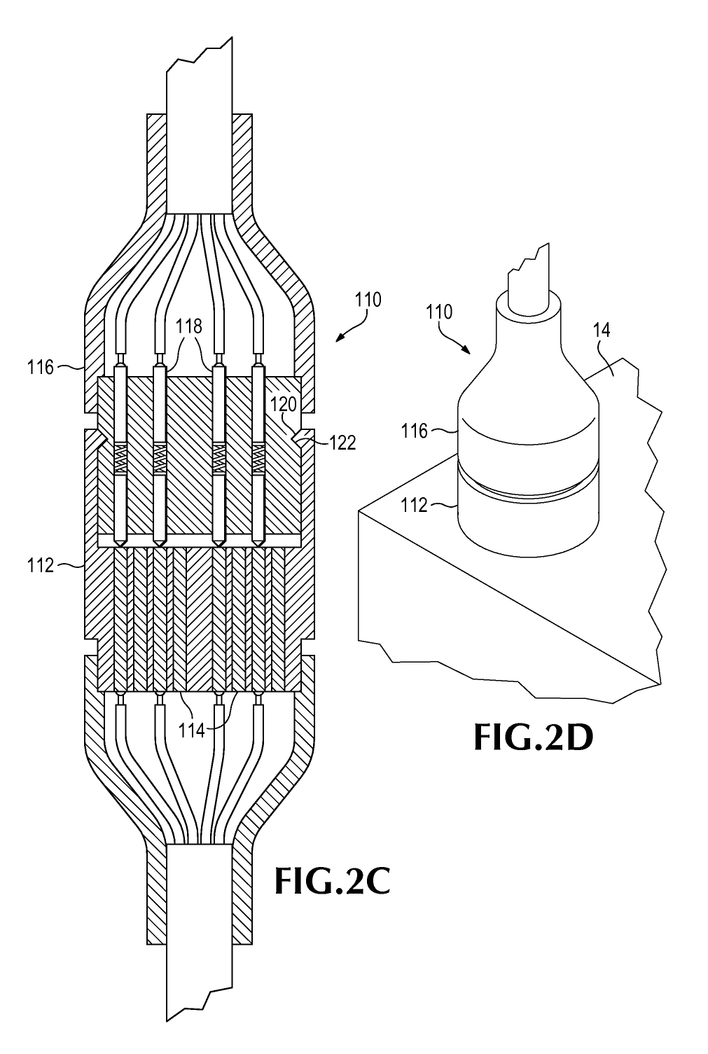

Referring to FIG. 1, a medical device assembly 10 is made up of a handheld medical device 12 connected to a base station 14 by a multi-conductor cable 16. A connector 18 is located either at the point where cable 16 meets base station 14, with one half of the connected being a part of base station 14 (FIG. 2D), or is located between two longitudinal halves of cable 16.

Each one of the following embodiments is shown with only a few contacts for ease of illustration. In reality, however, connectors according to the present invention may have upwards of fifty contacts, which would be necessary to support some of the hand-held devices available today. The contacts are typically have a surface layer of gold plated on nickel, which is plated onto copper. Nickel is used primarily to gain good adhesion of the gold, which does not bond well directly on copper. Gold is used because it does not oxidize. Oxidation could defeat the formation of robustly conductive connection between contacts. Another material that can be used for the contacts is a platinum-iridium alloy.

Referring to FIGS. 2A-2C, in one preferred embodiment a rotatable connector 110 is made up of a first half 112 defining a set of circular contacts 114, arranged concentrically. A second half 116 is made up of a set of spring-loaded, conductive pins 118 (pogo pins, in industry parlance), which are positioned so that each one will touch a circular contact 114 when the first and second halves are joined, thereby forming an electrical connection. when the second half 116 is rotated relative to the first half 112, the pins 118 move in a circle, with each pin maintaining contact with its corresponding circular contact 114. A lip defined by the housing for connector-half 112 fits into a groove 122 in the exterior of connector-half 116, to keep halves 112 and 116 together, but without fitting so tightly as to prevent rotation between the two halves, 112 and 116.

Various techniques may be used in constructing the connector described above. One method of creating concentric circle contacts 114 utilizes conductor deposition techniques used for printed circuit boards. In addition pogo-pins 118, other types of resilient contacts can be made, for example by a wire forming process in which the wire-end is compressed.

In an alternative preferred embodiment (not shown) each circular contact is broken up into a pair of semicircular contacts, with a pin connecting to each one.

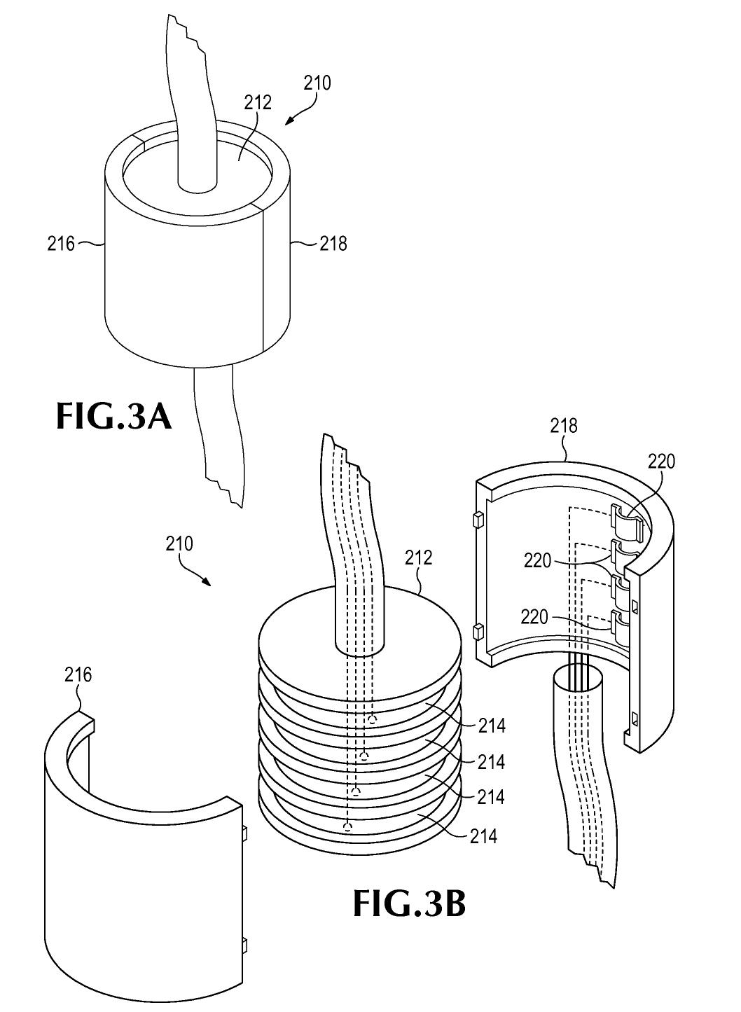

This alternative embodiment provides twice as many connections, but permits only 180 degree rotation. As noted previously, FIG. 2D shows the case in which half 112 of connector 110 is embedded in base station 14. Referring to FIGS. 3A and 3B, in an alternative preferred embodiment of a rotatable multi-contact connector 210, a first half 212 includes a set of circular contacts 214 arranged in stacked form. A second half includes first and a second semi-circular elements 216 and 218, adapted to lock together about first half 212. Element 218 has stacked resilient contacts 220 adapted to touch stacked circular contacts 214.

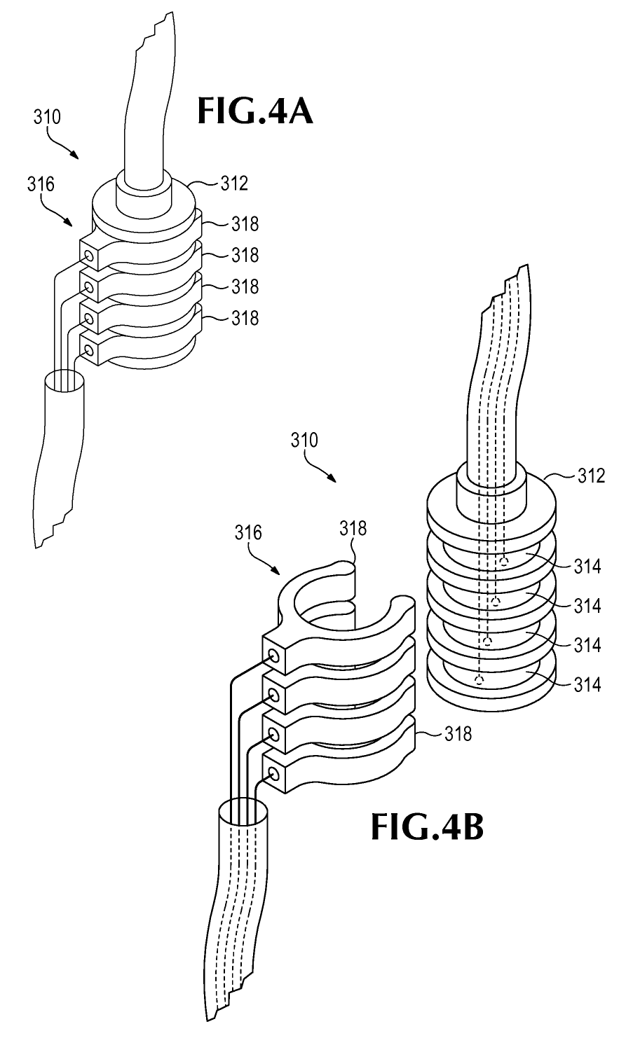

Referring to FIG. 4A and 4B, a further alternative preferred embodiment of a rotatable multi-conductor connector 310 has a first half 312 similar to first half 212 with stacked circular contacts 314, but wherein a second half 316 has a plurality of resilient horseshoe contacts 318, each being sufficiently flexible to snap about the corresponding circular contact 314. To gain this flexible contacts 318 may be formed of a flexible beryllium copper alloy and may have a thickness of about one millimeter.

In one preferred embodiment circular contacts 214 and/or 314 are made in modular fashion so that they can be easily fit together to form a connector having as many contacts as is desired.

One type of problem potentially encountered by the above described systems is that of a distortion of delicate analog signals caused by a variation in the robustness of the connection between two corresponding contacts whether a pin 118 with a circular contact 114, or a resilient contact 220 with a circular contact 214. One method of addressing this problem is to have a plurality of pins 118 or resilient contacts 220 per corresponding contact 114 or 214. The embodiment of FIGS. 4A and 4B, each arm of each horseshoe contact 318, acts largely as an independent contact, ensuring good connectivity.

In this manner, for a reduction in overall conductivity to occur in a signal path, at least two contact-to-contact paths would have to lose conductivity simultaneously. This amounts to at least two independent events, both of which are fairly rare. If for example, there was a 0.05 chance of either of two contact pairs falling below 50% of normal conductivity, then the chance of both falling below 50% at the same time would be 0.0025.

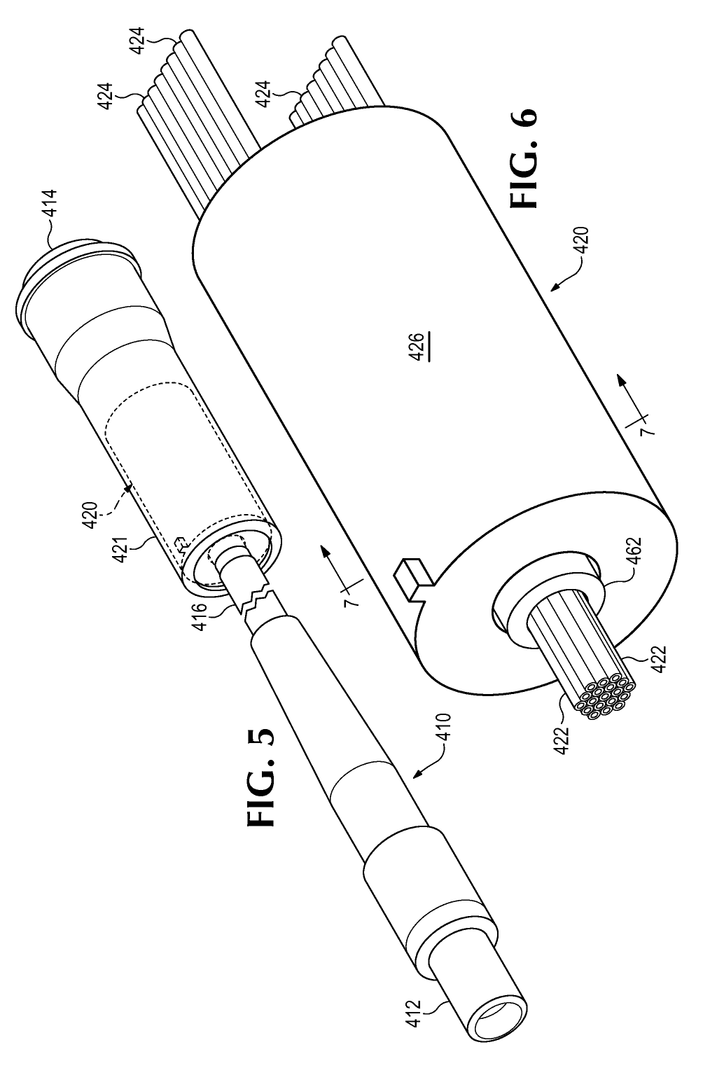

Referring to FIG. 5, the present invention may alternatively take the form of an adapter 410 that can be plugged in between a first cable section, using first connector-half 412, and a second cable section, using second connector half 414, to make the first cable section rotatable relative to the second section. A cable section 416 provides flexibility between first connector-half 412 and a rotation-permitting cylinder 420, which is held within an outer cylindrical housing 421.

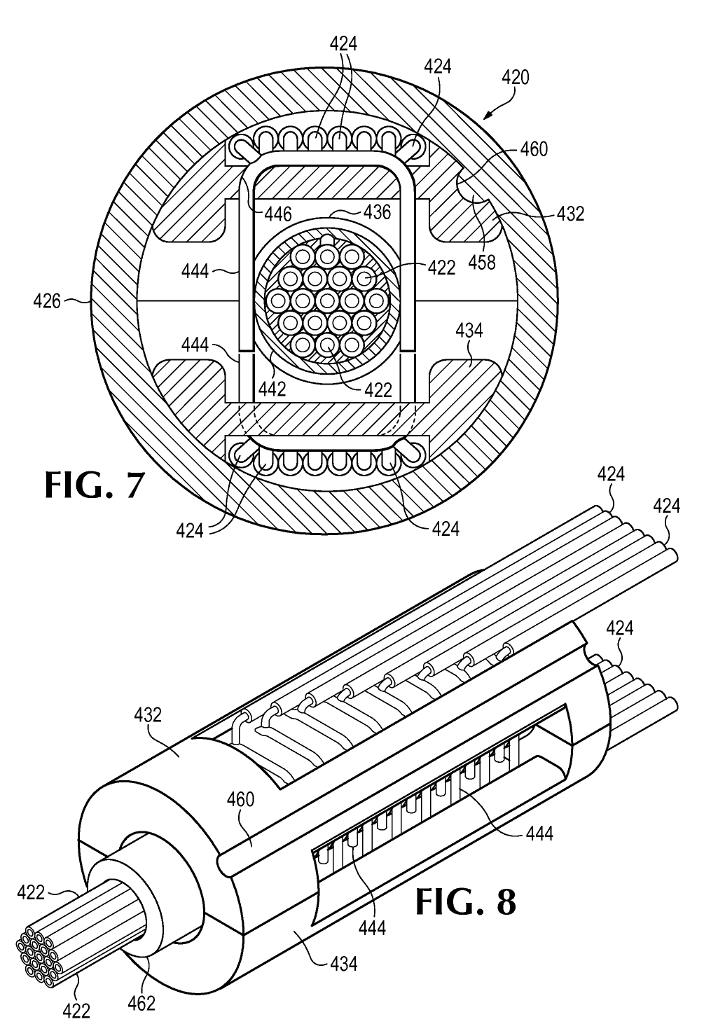

Referring to FIG. 6-9, rotation-permitting cylinder 420, permits first connector-half 412 to rotate relative to second connector-half 414, without interrupting the flow of electrical signals through adapter 410. A set of first wires 422 enters cylinder 420 from first connector-half 412 and a set of second wires 424 enters cylinder 420 from the second connector-half 414. An inner cylindrical housing 426 supports and protects inner portions of cylinder 420 described below.

Cylinder 420 includes a top frame 432 and a bottom frame 434 that fit together about a cylinder 436, which is permitted to rotate relative to the frame formed from upper and bottom frames 432 and 434. Each first wire 422 is electrically connected to a conductive ring 442 and each second wire is electrically connected to a conductive staple 444, which is held by a pair of apertures 446 in frame 432 or 434 so as to be in electrical contact with a ring 442. In FIG. 8, top and bottom stapes 444 are transversely aligned, with staples 444 extending downwardly from the top only extending part way down in the side window

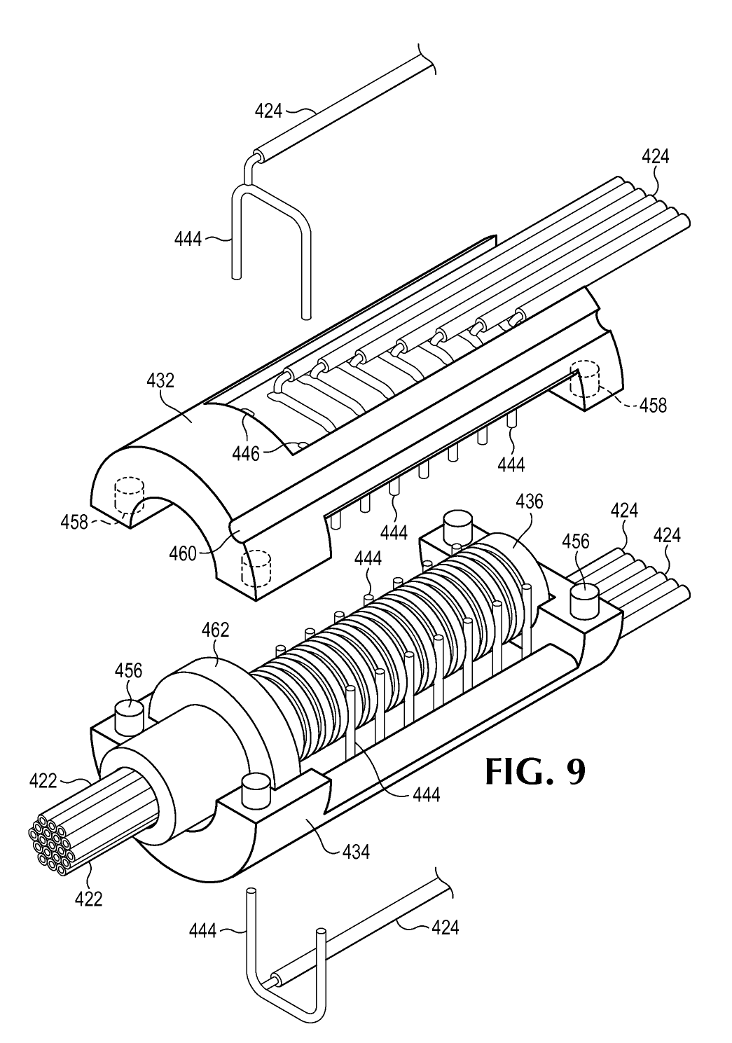

Top and bottom frames 432 and 434 are held together by pegs 456, which fit into matching holes 458. Further, frames 432 and 434 are oriented relative to inner cylindrical housing 426 by a ridge 458 (FIG. 7) that mates into a groove 460. A toroid bearing 462 (FIG. 9), helps to maintain the cylinder 436, in position relative to top and bottom frames 432 and 434.

In operation, cylinder 436 and therefore rings 442 are free to rotate relative to staples 444. Moreover, staples 444 are resilient and are held by frames 432 and 434 so as to press gently against rings 442, thereby providing a robust electrical contact. Both staples and rings are made of gold plated brass or beryllium copper.

Cylinder 436 is created by injecting polymer into a mold, with rings in place in the mold at the time the polymer is injected. In a preferred embodiment the polymer used is a low friction material such as Nylon. The first and second connector halves 412 and 414 are industry standard Redel® connectors. Cylinder 420 has a diameter 1.4 cm (0.55 in) and a length of 2.54 cm (1 in).

While a number of exemplary aspects and embodiments have been discussed above, those possessed of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A method of performing a surgical procedure using an electrical surgical device, comprising:
(a) providing an electrical surgical assembly including said surgical device connected to a base station by a cable having a plurality of mutually electrically insulated conductors, and an electrical adapter interposed at a point between said base station and said device;
(b) wherein said adapter is comprised of a first half and a second half that have freedom of rotation relative to each other, and wherein said first half is equipped with a first connector-half and said second half is equipped with a second connector-half; and
(c) performing said surgical procedure and permitting said adapter to permit said relative rotation thereby avoiding a problem of cable twisting.

2. The method of claim 1, wherein said first half of said adapter is connected directly to said base station by said first connector-half.

3. The method of claim 1, wherein said surgical device is selected from the group consisting of an ablation catheter, an intravenous ultrasound catheter, and an electrophysiology mapping catheter.

4. The method of claim 1, wherein said surgical device is an electric scalpel.

5. The method of claim 1, wherein said cable comprises a first cable portion and a second cable portion, in addition to said adapter, said first cable portion having a third connector-half mated to said first connector-half and said second cable portion having a fourth connector-half mated to said second connector-half, so that said adapter is interposed between said first cable portion and said second cable portion.

6. An electrical medical device assembly, comprising:
(a) a handheld unit, adapted to be manipulated by a medical professional and requiring multi-conductor electrical connection to a base unit;
(b) a base unit adapted to provide electrical power to said handheld unit;
(c) a multi-conductor electrical cable connecting said handheld unit to said base unit; and
(d) a multi-conductor electrical adapter interposed between said handheld unit and said base unit, said adapter including a first half and a second half and wherein relative rotation is permitted between said first half and said second half and wherein said first half is equipped with a first connector-half and said second half is equipped with a second connector-half.

7. The assembly of claim 6, wherein said first half of said adapter is electrically connected to said base station by way of said first connector-half.

8. The assembly of claim 6, wherein said surgical device is selected from the group consisting of an ablation catheter, intravenous ultrasound catheter, and an electrophysiology mapping catheter.

9. The assembly of claim 6, wherein said surgical device is an electric scalpel.

10. The assembly of claim 6, wherein said cable comprises a first cable portion and a second cable portion, in addition to said adapter, said first cable portion having a third connector-half mated to said first connector-half and said second cable portion having a fourth connector-half mated to said second connector-half, so that said adapter is interposed between said first cable portion and said second cable portion.

11. The assembly of claim 6, wherein said first half of said adapter defines a set of contacts having circular conductive surfaces and wherein said second half of said adapter includes a set of resilient contacts, each positioned to contact one said circular conductive surfaces to create an electrical connection.

12. The assembly of claim 11, wherein each of said circular conductive surfaces is contacted by a single resilient contact.

13. The assembly of claim 11, wherein said circular conductive surfaces are stacked and said resilient contacts are U-shaped elements.

14. The assembly of claim 13, wherein said second half of said connector fits around said first half.

15. The assembly of claim 11, wherein said first connector half includes an molded polymeric cylinder having embedded conductive ring contacts therein.

16. An adapter comprising:
(a) a first half that defines a plurality of contacts having circular conductive surfaces; and
(b) a second half that includes a set of resilient contacts, each positioned to contact one of said circular conductive surfaces to create an electrical connection.

17. The adapter of claim 16, wherein said second half of said adapter fits around said first half.

18. The adapter of claim 17, wherein said circular conductive surfaces are stacked and said resilient contacts are resilient U-shaped elements.

19. The adapter of claim 17, wherein said first half includes an injection molded polymeric cylinder having embedded conductive ring contacts.
